(19) United States
(12) Patent Application Publication
(10) Pub. No.: US 2025/0099784 A1
(43) Pub. Date: Mar. 27, 2025

(54) RADIOTHERAPY SYSTEM AND RELATED METHOD

(71) Applicant: Kongsberg Beam Technology AS, Kongsberg (NO)

(72) Inventors: Per Havard Kleven, Kongsberg (NO); Yapi Donatien Achou, Kongsberg (NO); Marcia Vagos, Oslo (NO); Mari Engebretsen Ryttervold, Oslo (NO); Karsten Ryden-Eilertsen, Borgen (NO)

(73) Assignee: Kongsberg Beam Technology AS, Kongsberg (NO)

(21) Appl. No.: 18/833,938

(22) PCT Filed: Feb. 10, 2023
(86) PCT No.: PCT/EP2023/053332

§ 371 Date: Jul. 29, 2024

(30) Foreign Application Priority Data
Feb. 11, 2022 (SE) 2230041-2

(51) Int. Cl. A61N 5/10; G06T 17/20; G16H 30/40

(52) U.S. Cl. CPC A61N 5/1049; A61N 5/103; A61N 5/1071; G06T 17/20; G16H 30/40; G06T 2210/41

(57) ABSTRACT

A method of establishing a biomechanical model (300) of an anatomical region (202) of a radiotherapy patient (200) for use in a radiotherapy system (100) is disclosed. The method comprising the steps of establishing a point-based or mesh-based model of the modelled anatomical region; associating static and/or dynamic features to points or mesh nodes (302) of the point-based or mesh-based model, the features comprising a world-space coordinate of the points or mesh nodes and state variable representing spatial-temporal displacement of the anatomical region and, for each point or mesh node, at least one feature characterising body tissue in the anatomical region; representing the model as a graph in a graph neural network system (500); and training the model on a cyclic or semi-cyclic motion of the anatomical region. A radiotherapy system is also disclosed.

### TECHNICAL FIELD

The present disclosure relates to a radiotherapy system comprising a control system for controlling an ionizing radiation beam supervising system. In particular, the present disclosure relates to predict movement, in particular cyclic or semi-cyclic movement, of the anatomical region to improve accuracy of dose delivery.

### BACKGROUND

Treatment of diseases using external beam radiotherapy, e.g. cancer treatment, involves applying ionising radiation to a patient so that radiation energy is deposited in malignant cells of the patient's body. If sufficient amount of energy is deposited, disruption of DNA and the subsequent death of the radiated cells may result.

Ionizing radiation can be directly or non-directly ionizing. Directly ionizing radiation utilises charged particles, e.g. electrons, protons, a particles and heavy ions. Indirectly ionizing radiation utilises neutral particles, e.g. photons (X-rays and γ-rays) and neutrons. The present disclosure is applicable to both types of external beam radiotherapy, i.e. using either directly or non-directly ionizing radiation. In particular, the present disclosure is applicable to X-ray or proton radiotherapy.

Protons and other charged particles display a depth-dose curve which is suitable for radiotherapy. Due to their relatively large mass, protons and other charged particles have little lateral side scatter in tissue and, consequently, the particle beam can be focused on malign tissue, minimizing dose side-effects to surrounding healthy tissue. Particle beams may also more precisely target malign tissue using the Bragg peak effect, i.e. the tendency of charged particles to deposit a large part of its energy in the very last region of the trajectory of the particle beam.

US2016/0144201A1 discloses a method of creating a proton treatment plan comprising the steps of dividing volumes of interest into sub-volumes, applying dose constraints to the sub-volumes based on, inter alia, patient movement, finding one or more feasible configurations of the proton therapy system, and selecting a proton beam configuration that improves or optimizes one or more aspects of the proton therapy.

However, a problem associated with the proton therapy system of US2016/0144201A1, and other prior art external beam radiotherapy systems, is that patient movement during treatment makes it difficult for the system to accurately deliver the radiation dose to the intended position within the radiation target. In fact, even if the patient is restrained, organ movements within the patient's body may still make it difficult for prior art radiation therapy systems to accurately deliver the radiation dose, as will motions resulting from breathing and involuntary muscular activity, e.g. heart beats.

Therefore, when preparing a treatment plan using prior art systems, uncertainty associated with patient and/or organ movements during treatment need to be considered when dose delivery is planned. In practise, this uncertainty may result in that the operator of the system sets the system to deliver a lower overall dosage to avoid damaging healthy tissue. This, in turn, may result in a less efficient therapy than if the uncertainty did not exist.

In particular, in the current radiotherapy practice relatively large margins are added to tumour volumes in the chest and upper abdominal cavities to compensate for the effects of respiratory motion on tumour dose delivery. This may result in compromises to prescribed tumour doses as well as treatment plans that may adversely affect the treatment outcome and increase the incidence of radiation induced morbidity.

WO2014/116868A1 discloses a motion compensation system for tracking and compensating for patient motion during a medical imaging scan. The system comprises an optical marker comprising an optically visible pattern and a first and a second optical detector positioned to digitally image the optically visible pattern along a first line respectively a second line of sight. The system further comprises a tracking engine configured to determine a pose of the object in six degrees of freedom by analysing images from the first and second optical detectors; and a controller interface configured to generate tracking information based on the pose and to electronically transmit the tracking information to a scanner controller to enable compensation within a medical imaging scanner for object motion.

WO2014/116868A1 suggests that the disclosed methodology for tracking patient movement can be applied in the therapeutic context, for example to track patient movement, e.g. due to breathing, in order to direct a therapeutic radiation beam at a diseased tissue region while avoiding surrounding healthy tissue.

However, whereas the system disclosed in WO2014/116868A1 may be able to track patient movement to some degree by virtue of the system tracking the optical marker and assigning movement of the optical marker to that of the patient, the system does not account for dynamic deformation or warping of the tissue region on which the optical marker is affixed.

Biomechanical models predicting patient motion are known in the art. For example, such biomechanical models are discussed in articles Patient-Specific Biomechanical Model for the Prediction of Lung Motion From 4-D CT Images (2014) Fuerst et al., IEEE transactions on medical imaging 34. 10.1109/TMI.2014.2363611 and Sensitivity of tumor motion simulation accuracy to lung biomechanical modeling approaches and parameters (2015), Tehrani et al., Phys Med Biol. 2015 Nov. 21; 60(22):8833-49. doi: 10.1088/0031-9155/60/22/8833. Epub 2015 November 4. PMID: 26531324; PMCID: PMC4652597. However, known biomechanical models, e.g. those discussed in the above-mentioned articles, are time-consuming computationally and not suitable to be used in real-time during radiotherapy dose delivery.

### SUMMARY

With the above-mentioned problems in mind, an object of the present disclosure is to provide a radiotherapy system and a related systems and methods that may at least alleviate the above-mentioned problems and/or reduce uncertainty in target localization during dose delivery.

Another object of the present disclosure is to provide a radiotherapy method and system involving a beam supervising system allowing accurate dose delivery to an irradiation target.

Another object of the present disclosure is to provide a radiotherapy system comprising a beam supervising system in which the beam supervising system can accurately track an irradiation target.

Yet another object of the present disclosure is to provide a radiotherapy system in which irradiation of organs-at-risk can be avoided.

A further object of the present disclosure is to provide a radiotherapy system that allows latency of the beam supervising system to be at least partially compensated for.

Yet a further object of the present disclosure is to provide a method of establishing a model of an anatomical region of a radiotherapy patient for use in a radiotherapy system.

Also, an object of the present disclosure is to provide a computer implemented method of planning at least a part of a radiotherapy procedure to be carried out on an anatomical region of a radiotherapy patient.

The claimed invention is specified in the independent claims of this application.

Advantageous adaptations and versions of the claimed invention are specified in the independent claims.

As previously mentioned, motion of anatomical regions may be modelled using biomechanical models. The underlying partial differential equations of such models may be solved in space and time using numerical computation methods, such as Finite Element Analysis, Finite Differences or Finite Volume Method, etc. The computations may be performed iteratively using a time stepping scheme that allows obtaining an evolution of model states. However, as previously mentioned, such computations are slow and require substantial computational resources and, therefore, are not suitable to be used in real-time during radiotherapy dose delivery.

In the paper Learning to Simulate Complex Physics with Graph Networks, Sanchez-Gonzalez et al., arXiv:2002.09405v2 [cs.LG]14.09.2020 (available at https://arxiv.org/abs/2002.09405) graph network-based simulators (GNS) are discussed and identified to be able to accurately simulate a wide range of physical systems in which fluids, rigid solids, and deformable materials interact with one another.

In the above-mentioned paper, a particle-based representation of a physical system is adopted where each of the particles represents the state of the system. Physical dynamics are approximated by interactions among the particles, e.g. by exchanging energy and momentum among their neighbours. The particle-based simulation can be viewed as message-passing on a graph. The nodes correspond to particles, and the edges correspond to pairwise relations among particles, over which interactions are computed. A learnable simulator computes dynamics information with a parameterized function approximator, do, whose parameters can be optimized for some training objective. The parameterized function approximator has three steps—encoder, processor, decoder.

The encoder embeds the particle-based state representation as a input graph in which node embedding are learned functions of the particles' states, directed edges are added to create paths between particle nodes which have some potential interaction, and a graph-level embedding can be added to represent global properties.

The processor computes interactions among nodes via steps of learned message-passing to generate an output graph.

The decoder extracts dynamics information from the nodes of the output graph.

In the paper Learning mesh-based simulation with Graph Networks, Pfaff et al., arXiv:2010.03409v4 [cs.LG]18.06.202 (available at https://arxiv.org/abs/2010.03409) a framework for learning mesh-based simulations using graph neural networks implementing the ENCODER-PROCESSOR-DECODER architecture followed by an integrator is disclosed.

In the discussed framework, a state of a system at time t is described using a simulation mesh with nodes connected by mesh edges. Each node is associated with a reference mesh-space coordinate which spans the simulation mesh, and additional dynamical quantities that are to be modelled. In systems in which the mesh represents a moving and deforming surface or volume, the mesh contains an extra world-space coordinate describing the dynamic state of the mesh in 3D space, in addition to the fixed mesh-space coordinate.

The framework learns a forward model of the dynamic quantities of the mesh at time t+1 given a current mesh, i.e. the mesh at time t.

The encoder encodes the current mesh into a multigraph. Mesh nodes become graph nodes, and mesh edges become bidirectional mesh-edges in the graph. World edges are added to the graph to enable learning external dynamics. World edges are edges created between nodes that are in close spatial proximity, excluding those node pairs that are already connected by mesh edges. World edges enable connectivity to be modelled not only between adjacent mesh nodes but also between nodes that are spatially close in world space, yet distant in mesh space. Features are encoded into the graph nodes and edges.

A relative displacement vector is encoded in mesh space and its norm into the mesh edges. A relative world-space displacement vector and its norm are encoded into both mesh edges and world edges. Remaining dynamical features as well as a one-hot vector indicating node type, are provided as node features.

The processor consists of a plurality of identical message passing blocks. Each block contains a separate set of network parameters and is applied in sequence to the output of the previous block, updating the mesh edge, world edge, and node embeddings.

After the final processing step of the processor, the latent node features are transformed in the decoder into one or more output features. The output features can be interpreted as (higher-order) derivatives of the dynamical features and can be integrated to compute the next-step dynamical quantity. Additional output features may be used to make direct predictions of auxiliary quantities.

Finally, the output mesh nodes are updated to produce the mesh for time t+1.

The present disclosure is based on the finding that a graph network system, if adopted and implemented correctly, may be used as a base for modelling and predicting, in real-time during radiotherapy dose delivery, cyclic or semi-cyclic movement or motion of an anatomical region comprising an irradiation target, in particular respiratory movement or motion.

The predicted cyclic or semi-cyclic movement or motion of the anatomical region can be used to control an ionizing radiation beam during a radiotherapy session such that desired dose delivery in the anatomical region is realised. For example, alignment of the beam can be controlled based on the predicted movement or motion such that the irradiation target is continuously tracked. Additionally or alternatively, the beam can be blocked or shut down when the predicted movement or motion of the anatomical region indicates that the irradiation target is about to venture outside of the range or trajectory of the ionizing radiation beam and/or when the predicted movement or motion indicates that the ionizing radiation beam is about to intersect an organ-at-risk. Consequently, the present disclosure is applicable to radiotherapy systems in which the alignment of the ionizing radiation beam can be controlled as well as in radiotherapy systems in which the alignment of the ionizing radiation beam cannot be controlled, in which first case the beam tracking function and/or the beam blocking or beam shut down function may be used, and in which latter case only the beam blocking or beam shut down function may be used.

According to a first aspect, the present disclosure provides a method of establishing a model of an anatomical region of a radiotherapy patient for use in a radiotherapy system, the method comprising the steps of:
(a) establishing a point-based or mesh-based representation or model of the anatomical region;
(b) associating static and/or dynamic features to points or mesh nodes of the point-based or mesh-based representation or model, the features comprising a world-space coordinate of the points or mesh nodes and state variable representing spatial-temporal displacement of the anatomical region and, for each point or mesh node, at least one feature characterising body tissue in the anatomical region;
(c) representing the point-based or mesh-based representation or model as a graph in a graph neural network system; and
(d) training the point-based or mesh-based representation or model on a cyclic or semi-cyclic motion of the anatomical region.

The step of establishing a point-based or mesh-based representation or model of the anatomical region typically comprises modelling the anatomical region as interconnected points or as a mesh comprising interconnected mesh nodes, and the step of representing the point-based or mesh-based representation or model as a graph in a graph neural network system typically comprises adopting the particle-based or mesh-based representation of the anatomical region such that each of the particles or mesh nodes represents the state of the system and such that physical dynamics are approximated by interactions among the particles or mesh nodes.

The step of training the point-based or mesh-based representation or model on a cyclic or semi-cyclic motion of the anatomical region typically comprises training the graph neural network system on a cyclic or semi-cyclic motion of the anatomical region, i.e. training the graph neural network system to predict cyclic or semi-cyclic motion of the anatomical region.

The at least one feature characterising body tissue in the anatomical region may advantageously comprise at least one variable characterising interaction between the anatomical region and an ionizing radiation beam. In particular, the at least one feature characterising interaction between the anatomical region and the ionizing radiation beam may comprise at least one of: linear stopping power; relative stopping power; radiodensity and radiopacity.

The step of representing the model as a graph in a graph neural network system may comprise establishing graph nodes, graph edges and graph global elements having features reflecting the static and dynamic variables of the biomechanical model.

The graph network system may comprise an input, a graph processing neural network, an updater and an output, and the graph processing neural network may comprise an encoder, a processor and a decoder.

The encoder may be configured to receive a current state of the mesh-based model from the input and encode an input graph from the current state of the model. Also, the processor may be configured to process the input graph and provide an output graph having updated node and edge embeddings.

Furthermore, the decoder may be configured to decode the output graph extracting output features corresponding to the static and dynamical variables of the biomechanical model, and the updater may be configured to update the output features to produce an inferred future state of the mesh-based biomechanical model and provide the inferred state to the output.

The step of training the model on a cyclic or semi-cyclic motion of the anatomical region may comprise applying a loss function on the inferred states provided by the updater and on a real-world dataset on an anatomical region corresponding to the modelled anatomical region.

The real-world dataset may comprise at least one of: 4D computed tomography (4D-CT) scans, 4D magnetic resonance imaging (4D-MRI) and ultrasound imaging.

The real-world dataset may comprise an evolution of images covering the modelled anatomical region during a respiratory cycle.

The point-based or mesh-based model may incorporate at least one of: a linear elasticity model and a hyperelasticity model adopted to the physical characteristics of the anatomical region.

The method may comprise the steps of:
dividing the anatomical region into at least a first anatomical sub-region displaying a first set of physical properties and a second anatomical sub-region displaying a second set of physical properties being different from the first set of physical properties;
for the first anatomical sub-region, assigning the point-based or mesh-based model a first point-based or mesh-based sub-model adopted to the first set of physical properties; and
for the second anatomical sub-region, assigning the point-based or mesh-based model a second point-based or mesh-based sub-model adopted to the second set of physical properties.

The first and the second anatomical sub-region may share a common boundary and the method may comprise the step of assigning the first and the second point-based or mesh-based sub-model common boundary conditions in the common boundary.

The graph network system as such may be implemented according to any one of said papers Learning to Simulate Complex Physics with Graph Networks, Sanchez-Gonzalez et al., arXiv:2002.09405v2 and Learning mesh-based simulation with Graph Networks, Pfaff et al., arXiv:2010.03409v4.

According to a second aspect, the present disclosure provides a computer implemented method of planning at least a part of a radiotherapy procedure to be carried out on an anatomical region of a radiotherapy patient, comprising:
establishing a biomechanical model of the anatomical region (202) according to the method of the first aspect; and
assigning, to each point or mesh node, at least one of: planned dose and maximum allowed dose.

According to a third aspect, the present disclosure provides a method of providing a radiotherapy system comprising at least one beam source arranged for irradiating an ionizing radiation beam onto an irradiation target located in an anatomical region of a radiotherapy patient. The method comprises the steps of:
establishing a biomechanical model of the anatomical region;
training the model on a cyclic or semi-cyclic motion of the anatomical region;
implementing the trained model in a graph network system implemented in a control system of the radiotherapy system;
establishing, in the graph network system, a current state of the trained model based on data on a current position and/or on a current orientation in space of the anatomical region;
establishing, in the graph network system and based on the current state of the trained model, an inferred future state of the trained model; and
controlling the ionizing radiation beam of the at least one beam source based on the inferred future state.

Said step of controlling the ionizing radiation beam of the at least one beam source may comprise controlling alignment of the at least one beam source such that the ionizing radiation beam is brought to track the irradiation target based on the inferred future state.

Additionally or alternatively, said step of controlling the ionizing radiation beam of the at least one beam source may comprise blocking the ionizing radiation beam or shutting down the at least one beam source should the inferred future state indicate that the irradiation target is about to venture outside the trajectory of the ionizing radiation beam.

Additionally or alternatively, said step of controlling the ionizing radiation beam of the at least one beam source may comprise blocking the ionizing radiation beam or shutting down the at least one beam source should the inferred future state indicate that the trajectory of the ionizing radiation beam is at risk of intersecting an organ-at-risk.

Blocking the radiation beam may be realised by applying a gating function to a beam supervising system, i.e. to a sub-system of the radiotherapy system that controls the at least one beam source.

The cyclic or semi-cyclic motion may typically be respiratory motion.

The step of establishing the biomechanical model may comprise establishing a point-based or mesh-based model of the anatomical region and associating static and/or dynamic features to points or mesh nodes of the point-based or mesh-based model. The features may comprise a world-space coordinate of the point or mesh node and a state variable representing spatial-temporal displacement of the anatomical region. The state variable may be a displacement field, i.e. an assignment of displacement vectors for all points or mesh nodes in the anatomical region that are displaced from one state to another.

The step of implementing the trained model in the graph network system may comprise representing the trained model as a graph in the graph network system by establishing graph nodes, graph edges and graph global elements having features reflecting the static and dynamic variables of the trained model.

The step of establishing a current state of the trained model may comprise correlating the current state with data on the current position and/or orientation in space of the anatomical region provided by a monitoring system of the radiotherapy system configured to monitor the anatomical region.

The monitoring system may be configured to monitor at least one of: a position and/or an orientation in space of a boundary area of the anatomical region; and a position and/or an orientation in space of a fiducial marker in the anatomical region.

Said variables may comprise at least one variable characterising interaction between the anatomical region and an ionizing radiation beam, e.g. linear stopping power and/or radiopacity. Said variables may additionally or alternatively comprise at least one variable characterising at least one of: planned radiation dose; maximum allowed radiation dose; and accumulated radiation dose.

The step of providing the inferred future state of the trained model may comprise updating the variable characterising the accumulated radiation dose, and the step of controlling position and/or alignment of the at least one beam source and/or blocking the ionizing radiation beam may comprise instructing a beam supervising system of the radiotherapy system to position and align the at least one beam source such that the irradiation target receives the planned radiation dose without the maximum allowed radiation dose for other parts of the anatomical region is exceeded.

According to a fourth aspect, the present disclosure provides a radiotherapy system comprising:
at least one beam source arranged to provide an ionizing radiation beam to an irradiation target located in an anatomical region;
a beam supervising system configured to control the at least one beam source;
a monitoring system arranged to monitor the anatomical region and, optionally, the beam supervising system;
a control system comprising a computer-implemented graph network system in which a model of the anatomical region, trained on cyclic or semi-cyclic motion of the anatomical region, is implemented;
the graph network system being configured to establish a current state of the trained model based on data on a current position and/or on a current orientation in space of the anatomical region received from the monitoring system, and being configured to establish an inferred future state of the trained model based on the current state; and
the control system being configured to instruct the beam supervising system to control the ionizing radiation beam of the at least one beam source based on the inferred future state.

The control system may be configured to instruct the beam supervising system to control the ionizing radiation beam of the at least one beam source based on the inferred future state to bring the least one beam source to track the irradiation target.

Alternatively or additionally, the control system may be configured to instruct the beam supervising system to control the ionizing radiation beam of the at least one beam source based on the inferred future state to block the ionizing radiation beam of the at least one beam source or to shut down the at least one beam source should the inferred future state indicate that the irradiation target is about to venture outside the trajectory of the ionizing radiation beam.

Alternatively or additionally, the control system may be configured to instruct the beam supervising system to control the ionizing radiation beam of the at least one beam source based on the inferred future state to block the ionizing radiation beam of the at least one beam source or to shut down the at least one beam source should the inferred future state indicate that the trajectory of the ionizing radiation beam is at risk of intersecting an organ-at-risk.

The trained model may be a point-based or mesh-based model comprising static and/or dynamic features associated with points or mesh nodes of the point-based or mesh-based model, the features comprising a world-space coordinate of the points or mesh nodes and state variable representing spatial-temporal displacement of the anatomical region.

The graph network system may be configured to represent the trained model as a graph comprising graph nodes, graph edges and graph global elements having features reflecting the static and dynamic variables of the trained model.

The graph network system may be configured to correlate the established current state of the trained model with data received from the monitoring system.

The monitoring system may be configured to monitor at least one of: a position and/or an orientation in space of a boundary area of the anatomical region; and a position and/or an orientation in space of a fiducial marker in the anatomical region.

The variables may comprise at least one variable characterising interaction between the anatomical region and an ionizing radiation beam, and at least one variable characterising at least one of: planned radiation dose; maximum allowed radiation dose; and accumulated radiation dose. For each inferred future state of the trained model, the graph network system may be configured to update the variable characterising the accumulated radiation dose, and the control system may be configured to instruct the beam supervising system to control position and/or alignment of the at least one beam source such that the irradiation target receives a planned radiation dose without a maximum allowed radiation dose for other parts of the anatomical region is exceeded.

According to a fifth aspect, the present disclosure provides a radiotherapy system comprising:
at least one beam source arranged to deliver an ionizing radiation beam to an anatomical region comprising an irradiation target;
a beam supervising system configured to control position and/or alignment of the at least one beam source;
a monitoring system arranged to provide updated, current data on the position and/or orientation in space of the anatomical region;
a control system comprising a computer-implemented graph network system in which a model of the anatomical region trained on cyclic or semi-cyclic motion of the anatomical region is represented as a graph comprising nodes, edges and global elements and including static attributes reflecting static features of the anatomical region and dynamic attributes reflecting dynamic features of the anatomical region, the control system being configured to:
   provide, based on information received from the monitoring system, a current representation or state of the anatomical region;
   process the current representation or state in the graph network system to provide at least one inferred future representation or state of the anatomical region; and
   control the ionizing radiation beam of the at least one beam source based on the at least one inferred future representation or state of the anatomical region.

Consequently, the radiotherapy system provides the control system with predicted future values of the dynamic features of the anatomical region, thus allowing the beam supervising system to adopt alignment of the beam to account for predicted movement and deformation of the anatomical region, in particular movement and deformation of the anatomical region due to respiratory activity of the radiotherapy patient. This allows the radiotherapy system to accurately track the radiation target taking due account of surrounding tissue, in particular organs-at-risk. This also allows latency of the beam supervising system to be at least partially compensated for, in particular latency of a beam guiding sub-system of the beam supervising system.

Additionally, or alternatively, should the predicted future values of the dynamic features of the anatomical region indicate that the ionizing radiation beam may intersect an organ-at-risk, the control system may be configured to block the ionizing radiation beam or shut down the at least on beam source.

Additionally, or alternatively, should the predicted future values of the dynamic features of the anatomical region indicate that the irradiation target is about to venture outside the trajectory of the ionizing radiation beam, e.g. due to the motion of the anatomical region bringing the irradiation target outside the range of the at least one beam source, the control system may be configured to block the ionizing radiation beam or shut down the at least one beam source.

The graph network system may preferably be configured to provide at least one inferred future representation or state of the anatomical region recurrently during dose delivery, preferably with a period within the range of 20 to 100 ms, e.g. 50 ms.

The graph network system may comprise an encoder configured to encode an input graph from the current representation of the anatomical region; a processor comprising one or more graph network blocks and trained to process the input graph to perform message passing along graph edges and provide an output graph having updated node and edge embeddings; and a decoder configured to decode the output graph extracting at least one output feature from the output graph.

The graph network system as such may be implemented according to any one of said papers Learning to Simulate Complex Physics with Graph Networks, Sanchez-Gonzalez et al., arXiv:2002.09405v2 and Learning mesh-based simulation with Graph Networks, Pfaff et al., arXiv:2010.03409v4.

The features may advantageously comprise features characterising body tissue in the anatomical region. The tissue characteristics may comprise linear or relative stopping power and/or radiodensity or radiopacity.

The features may also include at least one of planned dose, accumulated dose and maximum allowed dose associated with each point or mesh node.

Information on the position and/or alignment of the beam source(s) may be provided to the control system. In addition to being based on information received from the imaging system, the current representation of the anatomical region may also be based on information on the alignment of the beam source. Then, if the dynamic features comprise absorbed dose, the inferred future representation of the anatomical region to be updated also with regard to spatial distribution of absorbed dose, thus allowing dose delivery and the beam alignment to be controlled based on a difference between planned and absorbed dose.

The representation of the anatomical region may be provided as a mesh comprising mesh nodes and mesh edges, in which case the static and dynamic attributes may reflect static and dynamic features of the mesh nodes.

According to a sixth aspect, the present disclosure provides a computer-implemented control system for controlling an ionizing radiation beam supervising system to track an irradiation target in an anatomical region of a radiotherapy patient during dose delivery and/or to block a ionizing radiation beam should the ionizing radiation beam be at risk of intersecting an organ-at-risk or should the irradiation target be about to venture outside the trajectory of the ionizing radiation beam, the control system comprising a computer-implemented neural network system for processing data representing the irradiation target, wherein the anatomical region (including the irradiation target) is modelled in the neural network system using a simulation mesh M^{t}=(V, E^{M}) comprising nodes V connected by mesh edges E^{M}, wherein each node vᵢ∈V is associated with:
an input configured to receive a current state M^{t} and, optionally, at least one previous state M^{t-1} of the simulation mesh;
a reference mesh-space coordinate uᵢ which spans the simulation mesh;
at least one dynamic quantity qᵢ; and
a world-space coordinate xᵢ describing the dynamic state of the simulation mesh in 3D space,
   wherein the neural network system comprises:
a graph processing neural network;
an updater; and
an output configured to provide at least one inferred dynamic state M^{t+1} of the simulation mesh to the beam supervising system,
   wherein the graph processing neural network comprises:
an encoder configured to encode an input graph G⁰ from the current simulation mesh M^{t};
a processor comprising one or more graph network blocks and trained to process the input graph G⁰ to perform message passing along mesh edges and world edges and provide an output graph G^{M} having updated node and edge embeddings; and
a decoder configured to decode the output graph G^{M} extracting at least one output feature pᵢ corresponding to the at least one dynamical quantity qᵢ;
   wherein the updater being configured to update the at least one dynamical quantity qᵢ^{t+1}=pᵢ+qᵢ^{t} to produce the at least one inferred dynamic state M^{t+1}.

According to a seventh aspect, the present disclosure provides a computer-implemented method of modelling cyclic or semi-cyclic movement of an anatomical region comprising an irradiation target, comprising the steps of:
providing a simulation mesh M^{t}=(V, E^{M}) comprising nodes V connected by mesh edges E^{M} describing the anatomical region, wherein each node vᵢ∈V is associated with a reference mesh-space coordinate uᵢ which spans the simulation mesh M^{t}, a world-space coordinate xᵢ describing the dynamic state of the simulation mesh M^{t} in 3D space and, optionally, at least one additional dynamical quantity qᵢ;
creating, for each pair of nodes not already connected in the simulation mesh and having a spatial proximity less than a predetermined value |xᵢ−xⱼ|<r_{W}, a world edge E^{W};
in an encoder of a graph network system, encoding a current simulation mesh M^{t} into a multigraph G=(V, E^{M}, E^{W}) by encoding, into each mesh edge eᵢⱼ^{M}∈E^{M}, a relative mesh-space displacement vector uᵢⱼ=uᵢ−uⱼ and its norm |uᵢⱼ|, and encoding, into each mesh edge eᵢⱼ^{M}∈E^{M} and into each world edge eᵢⱼ^{W}∈E^{W}, a relative world-space displacement vector xᵢⱼ=xᵢ−xⱼ and its norm |xᵢⱼ|, and providing, for each node vᵢ∈V, said optional at least one additional dynamical quantity (qᵢ) as a node feature;
in a processor of the graph network system comprising a plurality of identical massage-passing Graph Net blocks, updating each mesh edge, each world edge and each node embeddings

$${e}_{ij}^{′ M} ← {f}^{M} ( {e}_{ij}^{M} , {v}_{i} , {v}_{j} )$$

$${e}_{ij}^{′ W} ← {f}^{W} ( {e}_{ij}^{W} , {v}_{i} , {v}_{j} )$$

$${v}_{i}^{′} ← {f}^{V} ( {v}_{i} , {\sum }_{j} {e}_{ij}^{′ M} , {\sum }_{j} {e}_{ij}^{′ W} )$$

by computing interactions among nodes via a predetermined number (M) of steps of learned message-passing, generating a sequence of updated latent graphs G=(G₁, . . . , G_{M}), where G^{m+1}=GN^{m+1}(G^{m}) and returning a final updated graph G^{M}=PROCESSOR (G⁰);
in a decoder of the graph network system, transforming the node features v into one or more output features pᵢ; and
in an updater of the graph network system, computing next-step dynamical features qᵢ^{t+1} by integrating the output features qᵢ^{t+1}=pᵢ+qᵢ^{t}, and updating output mesh nodes using the next-step dynamical features qᵢ^{t+1} to produce the at least one inferred dynamic state M^{t+1}.

According to a eight aspect, the present disclosure provides a biomechanical model of an anatomical region comprising an irradiation target, which model is trained on cyclic or semi-cyclic motion in a trained graph network system.

The features of any one of the above-discussed aspects may be combined with features of any one of the other aspects.

The disclosed system and method involve an anatomical biomechanical model that represents and simulates the internal state of a patient, and a graph neural network that is trained on simulated biomechanical data to learn the internal dynamics of the patient.

The biomechanical model can be used to simulate movements and deformations of internal organs during breathing, which simulation can be used to predict the internal state of a patient during the course of radiation therapy or surgery.

The biomechanical model may rely on a set of meshes that are three-dimensional (3D) in space, each representing a single anatomical structure, or on a set of point clouds defining various physical domains (mesh-free), such as organs and tissues.

The biomechanical model may be mathematically defined by a set of dynamic and constitutive equations, as well as boundary conditions, parameters, and initial values.

The biomechanical model may be solved in space and time to yield a series of estimates of 3D deformation fields that represent dynamic internal motions of anatomic structures, and which can be used to predict the locations of radiation deposition.

The biomechanical model may be solved in space and time using numerical computation methods, such as Finite Element Analysis, Finite Differences or Finite Volume Method, etc.

The computations may be performed iteratively using a time stepping scheme that allows obtaining a near real-time evolution of model states during radiation delivery. Here, real-time means that the model states are updated with a high enough cadence to allow for changes in internal anatomy to be captured and registered with sufficient resolution.

High resolution simulations are often very slow and, according to the present disclosure, learned models can be used instead to provide faster predictions. Biomechanical modelling (mesh-based or particle-based) may be used to generate data to train a neural network, which can then be used in the real-time computation of patient tissue biomechanics.

The computational model may predict the 3D spatial coordinates of target tissue and tissue-at-risk, as well as spatially varying tissue parameters, such as Hounsfield Units (HUs) and/or relative stopping power (RSP), at a requested time step t in the future that can range from milliseconds to seconds. Spatially varying quantities, such as HUs and RSP, can be extracted from a patient reference CT image and assigned to the respective nodes in the 3D anatomical model, prior to simulation. Since the spatial resolution of the mesh or point cloud may be lower than the original CT image, each data node may represent a neighbourhood of values.

The term “model” in this context refers to a model that is able to simulate (and predict) displacements over space and time, given a set of input parameters and a description of the system dynamics. The term “3D anatomical model” may refer to a geometrical representation of anatomical structures, or of an entire patient. The anatomical region may be the thorax, abdomen, head and neck or brain. The anatomical structure may be a solid cancerous or non-cancerous tumour, an organ, tissue, or a combination of those. The proposed motion prediction method is applicable to any structure undergoing cyclic or semi-cyclic translation, rotation, and/or deformations.

### Biomechanical Modelling

A dynamical model of physical domains can be broadly defined by a system of partial differential equations F with source terms f, boundary conditions g and h, and model parameters p. The model can also comprise of a set of constitutive equations and intermediate variables. A model state variable u may be used to represent spatial-temporal displacements of the domain Ω. t represents the time dimension.

$$F ( u , {u}^{′} , {u}^{″} , f , p , t ) = 0 , in Ω ⊂ {ℝ}^{n}$$

$$u ( x , t ) = g ( x , t ) , ∀ x in \partial Ω$$

$$\frac{\partial u ( x , t )}{\partial n} = h ( x , t ) , ∀ x in \partial Ω$$

$$f ( x , t ) , g ( x , t ) , h ( x , t ) : {ℝ}^{n} ↦ ℝ$$

$$p ⊂ ℝ$$

In the disclosed systems and methods, the state variable u may advantageously be a displacement field, i.e. an assignment of displacement vectors for all points in the domain that is displaced from one state to another. A displacement vector specifies the position of a point or a particle in reference to an origin or to a previous position. Consequently, in the disclosed systems and methods, a displacement field may be used to describe the effects of deformation on the anatomical region (including the irradiation target).

The mathematical model above is defined in such a way that it reproduces the relevant dynamics of the physical domains (in this case, quantified as u) within a certain accuracy range, and is constrained by a set of boundary conditions and model parameters which represent both physical properties and latent variables.

Each separate physical domain may be modelled by a different set of equations, and therefore, construction of such models may require expert knowledge of the physics and the mechanics of the corresponding domains. The requirement is that the model is capable of producing spatially and temporally resolved displacements of the domain nodes. The same boundary conditions may be applied to domains that share boundaries.

Said biomechanical model may be based on the above-discussed mathematical model or on any dynamical model that is formulated as a forward map that integrates the state variable u from time t to t+1.

### Elasticity Model

A number of mathematical models exist that describe the behaviour of material, including:
Linear elasticity model: Linear elastics describe the deformation of a body under an external or internal load as being linearly proportional to the load. Hooke's law represents the material behaviour and relates the unknown stresses and strains.
Hyperelasticity model: These materials show not only the nonlinear material behaviour, but also the large deformation and stress-strain relationship are derived from a strain energy density function. Examples of hyperelastic material models are the Neo-Hookean and Mooney-Rivlin model.

Said biomechanical model may incorporate any of these mathematical models.

### Mesh-Based and Meshless Methods

The biomechanical 3D anatomical model may be represented either as a tetrahedral mesh or a cloud of points/particles, for which different numerical methods may be employed to solve the underlying system of partial differential equations.

### Meshless/Particle-Based Methods

Meshless methods may utilize an unstructured cloud of points for spatial discretization. The collocation of nodes within the domain can either be arbitrary, or follow certain rules that ensure, for instance, that a part of a domain contains a higher density of nodes, thus allowing for a higher accuracy of the solution in that subdomain.

### Mesh-Based Methods

Numerical simulation in biomechanics is commonly solved by the Finite Element Method (FEM). The domains of independent organs/tissues may be discretized into a finite set of interconnected nodes and cells on which the solution of the finite element analysis may be computed. The meshes may be generated from organ and tumour segmentation to divide the structures into small connected tetrahedron elements.

### Domains

A domain may be regarded as a physical continuum that can be described by the same set of equations, parameters, and constraints. The physical domains of the biomechanical model may be the anatomical structures of interest, which typically include the tumour or tumours to be treated, the organ in which it is formed, and surrounding organs-at-risk (OAR). They can also include non-OARs and the outer surface of the body.

The domains may be partitioned into a finite number of cells or nodes, resulting in either a mesh or a point cloud, which may serve as a basis for model discretization in the scheme of numerically solving the biomechanical model.

### Boundary Conditions

Boundary conditions may be defined prior to simulation in order to constrain the biomechanical model to a unique solution. In the case of a mesh-based model, boundary conditions may be defined on the facets of each domain (boundary). The boundary conditions may either prescribe a desired value of the state variable on the boundary (Dirichlet boundary condition), or the gradient of the state variable (Neumann boundary condition), or a combination of both (Robin, mixed and Cauchy boundary conditions). Since the state variable is the displacement, which is a vector quantity, boundary conditions are also a vector quantity. Boundary conditions can then be obtained by calculating the deformations from one image (the reference image) to the other (the moving image). Therefore, in order to obtain boundary conditions from images, one needs at least two images of the patient from the same imaging study taken at different times.

Boundary conditions can also be obtained from alternative data sources other than imaging, for instance, from monitoring sensors, in which case the boundary conditions will typically be used to constrain a subregion of the boundary.

Boundary conditions may be patient-specific, that is, they may be derived from the patient's own imaging data, or they may be derived from other patient's data.

### Internal Forces

Forces and pressures on internal organs driven by for example muscular movements, respiration, bolus, and bladder filling, may be embedded into the biomechanical model by including forcing terms (also called source terms) into the differential equations. These forces can be used to impose further constraints on the model to behave in a physiological (as well as patient-specific) manner, and thus may be tuned to the application.

### Solving the Biomechanical Model

The biomechanical model may be solved using any suitable FEM software.

### Graph Network Framework

The methods and systems according to the disclosure use a graph network framework or system to predict future states of the biomechanical model, e.g. a framework based on the teaching of paper Relational inductive biases, deep learning, and graph networks, Battaglia et al., arXiv:1806.01261v3 [cs.LG]17.10.2018 (available at https://arxiv.org/abs/1806.01261).

The methods and systems according to the disclosure also use a graph network framework or system to learn the dynamics of the biomechanical model.

Within the graph network framework or system, a graph may generally be defined as a 3-tuple G=(g, V, E), where entities are represented by the graph's nodes V, relations by the graph's edges E, and system-level properties by global attributes g.

The main unit of computation in the graph network framework or system is the graph network block (GN block). The graph network block is a “graph-to-graph” module which takes a graph as input, performs computations over the structure, and returns a graph as output.

The graph network block contains three sub-functions—one for each of the edge E, the node V and the global attributes g. The sub-functions can be implemented by potentially using any class of neural network. Each of these functions may be implemented with, for example, a multilayer perceptron (MLP), a recurrent neural network (RNN), a long short-term memory (LSTM) recurrent neural network or a convolutional neural network (CNN).

Implementations of the sub-functions may be based on DeepMind's Sonnet library (https://github.com/deepmind/sonnet).

If the 3D biomechanical model is based on a mesh, the nodes and edges of the graph may be specified to represent the nodes and vertices of the mesh, respectively. The dynamic quantities of the system, i.e. the state variables and their derivatives, may be embedded in the edges of the graph. A processor of the graph network block takes the input graph and updates the dynamic quantities defined on the edges to produce the output graph.

The features received by each edge determine the physical interactions and properties from the biomechanical model that are encoded in the network.

The graph edges embed the relative displacement field u and its norm |u|. Additionally, internal forces in the model may be encoded as directional edge features between two adjacent nodes, representing the force exerted by one node onto the neighbouring node.

Differential operators may be approximated by message-passing between adjacent nodes, which are embedded in the edges of the graph as sub-functions (ƒ^{M}). These functions take the edge features (e.g. derivatives and forces) and the sender and receiver node features (coordinates, material properties, etc.) as input, and output the higher-order derivatives of the nodes. These are in turn used to integrate the dynamic variable x.

The graph nodes may also embed functions (ƒ^{v}) which represent operations on the graph node features, independent of the messages passed between graph nodes, e.g. transformations of the material properties.

$${e}_{ij}^{′ M} ← {f}^{M} ( {e}_{ij}^{M} , {v}_{i} , {v}_{j} )$$

$${v}_{i}^{′} ← {f}^{V} ( {v}_{i} , {\sum }_{j} {e}_{ij}^{′ M} , {\sum }_{j} {e}_{ij}^{′ W} )$$

Here, eᵢⱼ^{M} and eᵢⱼ^{W} are the mesh and world edges, respectively.

| Inputs | Inputs | Inputs | Outputs |
|---|---|---|---|
| eᵢⱼ^{M} | eᵢⱼ^{W} | vᵢ | pᵢ |
| uᵢⱼ, \|uᵢⱼ\|, xᵢⱼ, \|xᵢⱼ\| | xᵢⱼ, \|xᵢⱼ\| | nᵢ | Fᵢ, x′ᵢ |

The table below describes the features of the graph of the systems and methods according to one aspect of the present disclosure.

Node features (vᵢ)

| | |
|---|---|
| xᵢ | 3D coordinates of node vᵢ |
| x′ᵢ (for second order | First order derivative of xᵢ |
| models only) | |
| nᵢ | Node type |
| Nᵢ | Number of neighbours |

Edge features (eᵢⱼ)

| | |
|---|---|
| uᵢⱼ = xᵢ − xⱼ | Relative mesh-space displacement vector |
| | between node vᵢ and vⱼ. |
| \|uᵢⱼ\| | Norm of relative displacements |
| xᵢⱼ | Relative world-space displacement vector |
| | between node vᵢ and vⱼ. |
| fᵢⱼ | Internal forces |

Global features

| | |
|---|---|
| uₘₐₓ | Maximum allowed displacement (edge |
| | length) |
| V | Span of xᵢ |

Outputs

| | |
|---|---|
| x′ᵢ (for first-order | First-order derivative of xᵢ |
| models only) | |
| x″ᵢ (for second-order | Second-order derivative of xᵢ |
| models only) | |
| Fᵢ | Field variable (e.g. stress) |

For first-order models, the dynamic quantity xᵢ at time t+1 is obtained by integrating the derivatives at time t according to:

$${x}_{i}^{t + 1} = {x}_{i}^{′} + {x}_{i}^{t}$$

Consequently, for first-order models, the dynamic quantity xᵢ is updated with the first derivative, output by the graph network.

For second order models, the dynamic quantity xᵢ at time t+1 is obtained according to the following:

$${x}_{i}^{t + 1} = {x}_{i}^{″} + 2 {x}_{i}^{t} - {x}_{i}^{t - 1}$$

For second-order models, x′ᵢ is embedded as a node feature and x″ᵢ is used to integrate the dynamic quantity xᵢ.

In the following, the invention will be discussed in more detail with reference to the appended drawings.

### DESCRIPTION OF THE DRAWINGS

The following drawings and the associated descriptions are provided to illustrate embodiments of the disclosure and do not limit the scope of the claims.

FIGS. 1 and 2 schematically illustrate an embodiment of a radiotherapy system according to the present disclosure.

FIG. 3. illustrates an anatomical region of a radiotherapy patient.

FIG. 4 illustrates a mesh-based model of the anatomical region according to FIG. 3.

FIG. 5 illustrates a graph encoded from the mesh-based model according to FIG. 4.

FIG. 6 illustrates a graph network system.

FIG. 7 illustrates graph network blocks.

FIG. 8 illustrates an embodiment of a method of establishing a biomechanical model of an anatomical region of a radiotherapy patient for use in a radiotherapy system.

FIG. 9 illustrates an embodiment of a computer implemented method of planning at least a part of a radiotherapy procedure to be carried out on an anatomical region of a radiotherapy patient.

### DETAILED DESCRIPTION

An embodiment of radiotherapy system according to the present disclosure, and how such a system is provided, will in the following be discussed in more detail within a setting in which the radiotherapy system 100 is configured to predict and compensate for respiratory motion of a radiotherapy patient during dose delivery. It is to be understood, however, that the following teaching may be applied to predict and compensate for any type of cyclic or semi-cyclic motion.

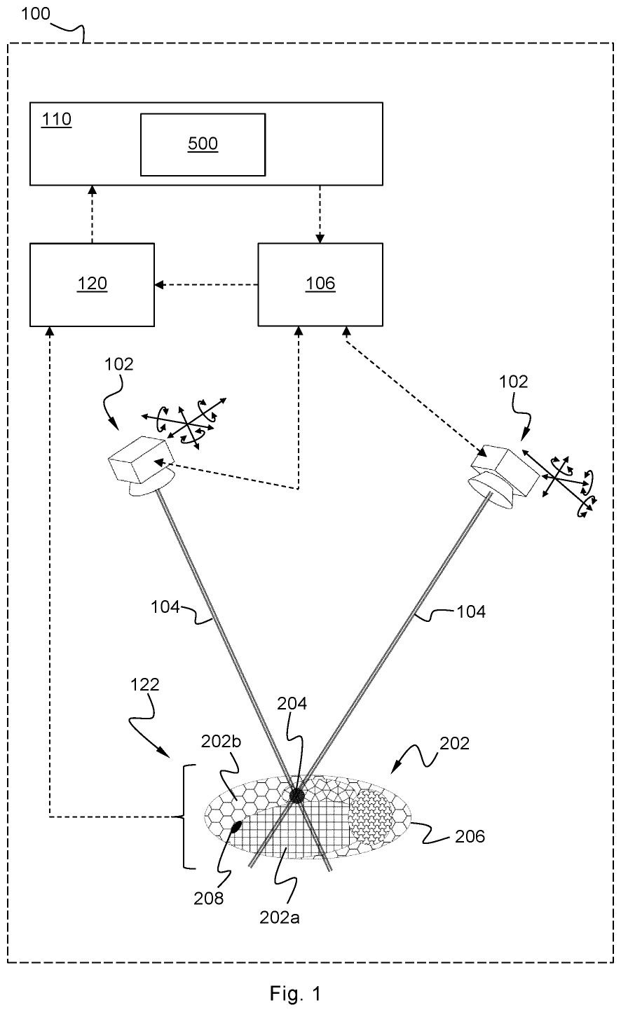

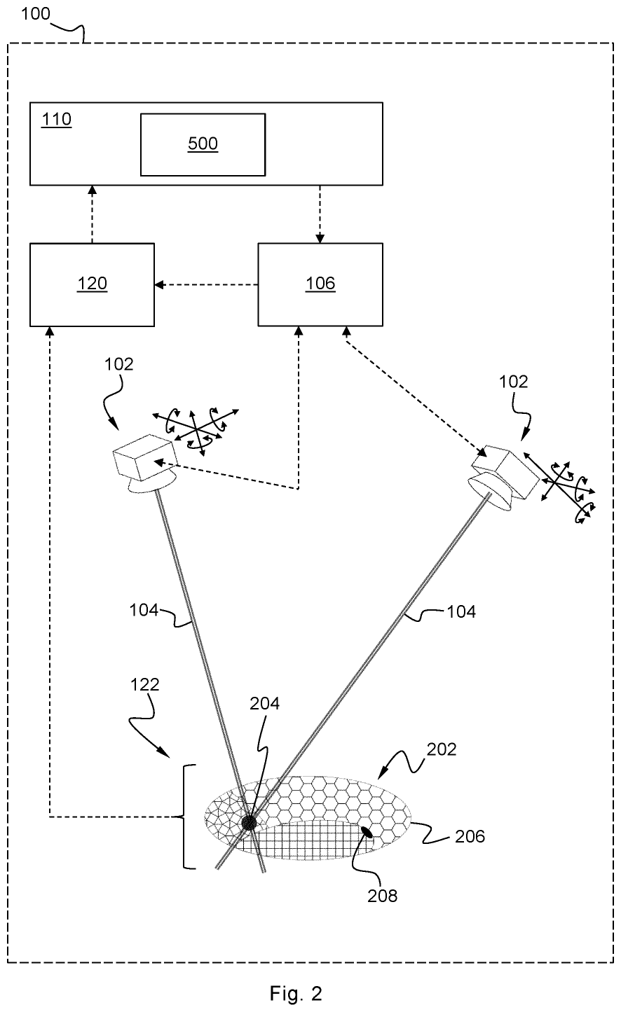

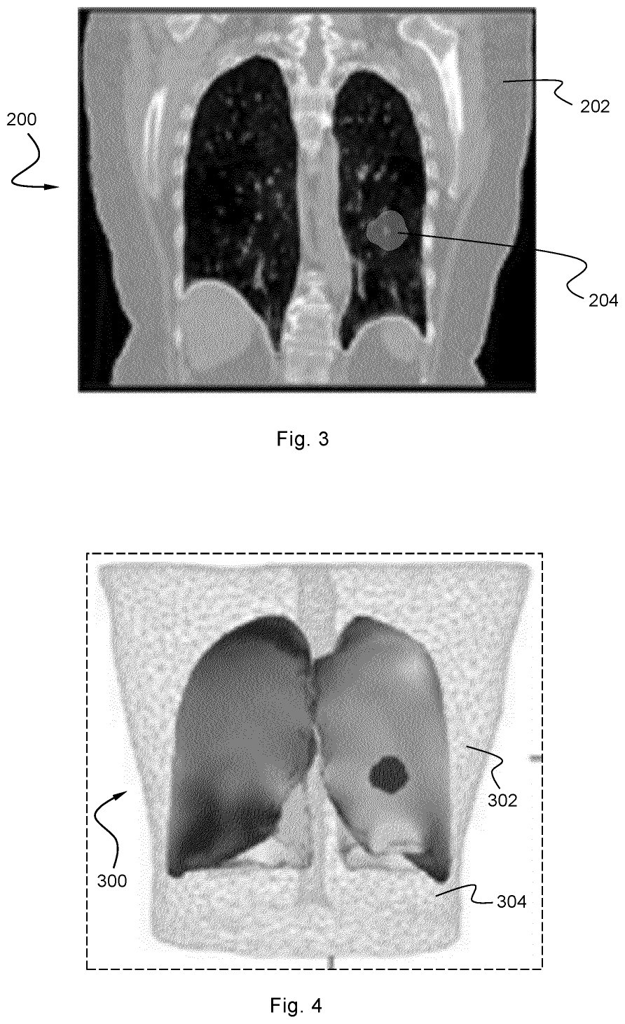

FIGS. 1 and 2 schematically illustrate an embodiment of a radiotherapy system 100 according to the present disclosure. The radiotherapy system 100 is configured to irradiate an irradiation target in an anatomical region of a radiotherapy patient. FIG. 3 is an illustration of a radiotherapy patient 200 having an anatomical region 202, in this case a thorax, in which there is an irradiation target 204. In FIGS. 1 and 2, the anatomical region and the irradiation target are schematically illustrated by reference numerals 202 and 204, respectively.

The anatomical region 202 comprises different anatomical structures having different tissue characteristics, some of which structures may be organs-at-risk, OAR, i.e. structures or organs which should preferably not be irradiated during radiotherapy treatment. The irradiation target 204 may typically be a tumour.

Providing the radiotherapy system 100 involves establishing, prior to a radiotherapy session, a 3D mesh-based biomechanical model of the anatomical region 202 comprising mesh nodes and mesh vertices, and associating each mesh node with static and dynamic variables characterising properties of the anatomical region 202 in or at that mesh node, thus allowing states of the anatomical region 202 to be represented by a set of mesh node which encode properties within local regions of space of the anatomical region 202.

FIG. 4 illustrates a biomechanical model 300 of the anatomical region 202 disclosed in FIG. 3, comprising mesh nodes 302 and mesh vertices 304, wherein each mesh node 302 is associated with a plurality of static and dynamic variables. The static and dynamic variables comprise a world-space coordinate x of the mesh node 302. The static and dynamic variables also comprise at state variable u that represents spatial-temporal displacement of the anatomical region 202. The state variable u may advantageously be a displacement field, i.e. an assignment of displacement vectors for all mesh nodes 302 in the anatomical region 202 that are displaced from one state to another. Generally, a displacement vector specifies the position of a point or a particle in reference to an origin or to a previous position. Consequently, a displacement field u may be used to describe the effects of deformation on the anatomical region and, consequently, also on the irradiation target.

Advantageously, the static and dynamic variables may also comprise at least one variable characterising interaction between the anatomical region 202 and an ionizing radiation beam in or at each mesh node 302 or voxel, e.g. linear stopping power or radiopacity, e.g. expressed as Hounsfield units. The static and dynamic variables may also comprise variables characterising planned and/or maximum allowed radiation dose in or at each mesh node 302. The static and dynamic variables may also comprise a variable characterising accumulated radiation dose in or at each mesh node 302.

The biomechanical model 300 may in principle be based on any mathematical model of the anatomical region that allows movements and deformations of organs and tissue in the anatomical region 202 to be simulated. The biomechanical model may be mathematically defined by a set of dynamic and constitutive partial differential equations, as well as boundary conditions, parameters, and initial values, which equations can be solved in space and time to yield a series of estimates of 3D deformation fields that represent dynamic internal motions of the anatomical region 202. Typically, such solving involves solving the equations in space and time using numerical computation methods, such as Finite Element Analysis, Finite Differences or Finite Volume Method, etc. Such computational methods may be performed iteratively using a time stepping scheme that allows an evolution of model states to be obtained which are updated with a high enough cadence to allow for changes in internal anatomy to be captured and registered with required resolution. Different sub-regions of the anatomical region 202 may be modelled with different mathematical models. In particular, different sub-regions may be modelled by a different set of equations taking into account the different tissue types present in biomechanical model 300.

For example, establishing the biomechanical model 300 may comprise dividing the anatomical region 202 into at least a first anatomical sub-region 202a displaying a first set of physical properties and a second anatomical sub-region 202b displaying a second set of physical properties being different from the first set of physical properties (see FIG. 1). For the first anatomical sub-region 202a, the mesh-based model 300 may be assigned a first mesh-based sub-model adopted to the first set of physical properties; and for the second anatomical sub-region 202b the mesh-based model 300 may be assigned a second mesh-based sub-model adopted to the second set of physical properties.

The first and the second anatomical sub-regions 202a, 202b may share a common boundary and the method of establishing the biomechanical model 300 may comprise the step of assigning the first and the second point-based or mesh-based sub-models common boundary conditions in the common boundary.

A number of mathematical models exist that describe the behaviour of material. These models include linear elasticity models, in which linear elastics describe the deformation of a body under an external or internal load as being linearly proportional to the load. In these models Hooke's law represents the material behaviour and relates the unknown stresses and strains. Known mathematical models also include hyperelasticity models, in which materials display not only nonlinear material behaviour, but also a large deformation and stress-strain relationship derived from a strain energy density function. Examples of hyperelastic material models include the Neo-Hookean model and the Mooney-Rivlin model.

Said biomechanical model 300 may incorporate any one of these mathematical models.

Generally, the biomechanical model 300 can be defined by a system of partial differential equations F with source terms f, boundary conditions g and h, and model parameters p:

$$F ( u , {u}^{′} , {u}^{″} , f , p , t ) = 0 , in Ω ⊂ {ℝ}^{n}$$

$$u ( x , t ) = g ( x , t ) , ∀ x in \partial Ω$$

$$\frac{\partial u ( x , t )}{\partial n} = h ( x , t ) , ∀ x in \partial Ω$$

$$f ( x , t ) , g ( x , t ) , h ( x , t ) : {ℝ}^{n} ↦ ℝ$$

$$p ⊂ ℝ$$

The model state variable u is the displacement field that represent spatial-temporal displacements of the domain SZ and t represents the time dimension.

Providing the radiotherapy system 100 further involves training the model on the cyclic or semi-cyclic motion the radiotherapy system 100 is configured to predict and compensate for, i.e. respiratory motion in the present embodiment.

Preferably, this training involves representing the model 300 as a graph in a graph network system and applying a loss function on inferred future states provided by the graph network system.

Representing the model 300 as a graph in a graph network system involves establishing graph nodes, graph edges and graph global elements having features reflecting the static and dynamic variables of the biomechanical model 300.

Graph networks are known as such, e.g. through the above-discussed papers Learning to Simulate Complex Physics with Graph Networks, Sanchez-Gonzalez et al., arXiv:2002.09405v2 and Learning mesh-based simulation with Graph Networks, Pfaff et al., arXiv:2010.03409v4, and the graph network according of the radiotherapy system 100 may advantageously be implemented taking as a starting point the teaching of any one of these papers.

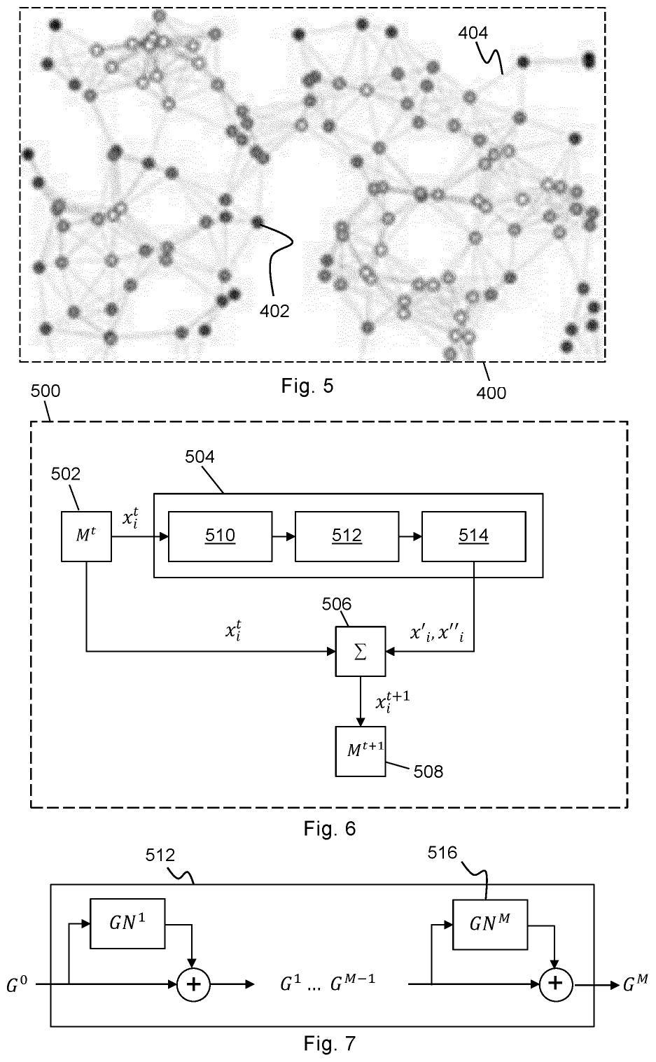

FIG. 5 schematically illustrates a graph 400 representing the biomechanical model 300 illustrated in FIG. 4, which graph comprises graph nodes 402 and graph edges 404. In the present embodiment, the graph nodes 402 and the graph edges 404 may be specified to represent the mesh nodes 302 and the mesh vertices 304, respectively, and the state variables representing the spatial-temporal displacement of the anatomical region 202, e.g. represented by a displacement field u, may be embedded in the graph edges 404. Additionally, internal forces within the biomechanical model 300 may be encoded as directional graph edge features between two adjacent graph nodes, representing the force exerted by one mesh node onto a neighbouring mesh node.

FIG. 6 schematically illustrates the main building blocks of a graph network system or framework 500 implemented within the context of the present embodiment of the radiotherapy system 100.

The graph network system 500 comprises an input 502, a graph processing neural network 504, an updater 506 and an output 508. The graph processing neural network 504 comprises an encoder 510, a processor 512 and a decoder 514.

The encoder 510 is configured to receive a current state M^{t} of the mesh-based biomechanical model 300 from the input 502 and encode an input graph G⁰ from the current state M^{t} of the biomechanical model 300.

The processor 512 is configured to process the input graph G⁰ and provide an output graph G^{M} having updated node and edge embeddings. The processor 512 comprises one or more graph network blocks 516 (see FIG. 7) which take a graph as input, performs computations over the graph structure, and returns a graph as output. Each graph network block contains three sub-functions—one for each of the graph edge, the graph node and the graph global elements. The sub-functions can be implemented by potentially using any class of neural network. Each of the sub-functions may be implemented with, for example, a multilayer perceptron (MLP), a recurrent neural network (RNN), a long short-term memory (LSTM) recurrent neural network or a convolutional neural network (CNN).

Implementations of the sub-functions may be based on DeepMind's Sonnet library (https://github.com/deepmind/sonnet).

The decoder 514 is configured to decode the output graph G^{M} extracting output features corresponding to the static and dynamical variables of the biomechanical model 300.

The updater 506 is configured to update the output features to produce an inferred future state M^{t+1} of the mesh-based biomechanical model 300 and provide the inferred state M^{t+1} to the output 508.

Training the biomechanical model 300 preferably involves applying a loss function on the inferred states provided by the updater and on a real-world dataset on an anatomical region corresponding to the modelled anatomical region 202. The real-world dataset may be based on 4D computed tomography (4D-CT) scans of the anatomical region corresponding to the modelled anatomical region 202. To this end, Hounsfield units, giving the opacity of a tissue material to X-rays, should preferably be included as a variable in the model 300. Alternatively, or additionally, training may be based on datasets acquired from 4D magnetic resonance imaging (4D-MRI) or ultrasound imaging.

For training respiratory motion, the dataset may preferably be based on an evolution of images covering the modelled anatomical region 202 during a respiratory cycle. To train the model, a minimum of two image datasets of the same patient could be used, each capturing different breathing patterns. Preferably, however, images from a larger population of patients, exhibiting different breathing patterns (inter-patient and intra-patient variations) could be used.

Referring now back to FIGS. 1 and 2, the radiotherapy system 100 comprises at least one beam source 102 arranged to provide an ionizing radiation beam 104. The radiation beam 104 may be a particle beam, e.g. a proton beam, or a photon beam, e.g. an X-ray beam.

The radiotherapy system 100 further comprises a beam supervising system 106 configured to control the position in space and the alignment of each beam source 102. This control may preferably comprise controlling three variables defining the position of each particle beam source, e.g. represented by Cartesian coordinates x, y, z, and/or variables defining pith, jaw and/or roll of the beam source, e.g. as represented by angles of rotation measured about orthogonal pitch, jaw and/or roll axes, as is schematically illustrated in FIGS. 1 and 2.

The radiotherapy system 100 also comprises a control system 110 which is configured to control the beam supervising system 106 to track the irradiation target 204. To this end, the control system 110 comprises a computer-implemented graph network system or framework 500 in which the above-discussed trained biomechanical model 300 is implemented. The graph network system 500 may advantageously be the same type of graph network system 500 as used for training the model 300.

The radiotherapy system 100 also comprises a monitoring system 120 arranged to monitor the anatomical region 202 to receive updated data on at least one aspect of the current state of the anatomical region 202. “Updated data” here refers to data that is sufficiently up-to-date to provide information on the current state of the anatomical region 202. Consequently, although the updated data should preferably be real-time or near real-time data, less recent data may qualify as updated data for cyclic or near-cyclic motion having slowly evolving states.

The monitoring system 120 may for example be configured to monitor the position and/or orientation in space of a boundary area 206 of the anatomical region 202, for example the envelope of the thorax of the radiotherapy patient, i.e. the outer contour (body surface) of the thorax, e.g. indicated by skin markers. However, monitored data may in addition or alternatively comprise the position and/or orientation in space of internal structures of the anatomical region, e.g. the position and/or orientation in space of one or a plurality of fiducial markers 208. This data may be received by the monitoring system 120 from an imaging system 122, e.g. a camera operating within the visible electromagnetic spectrum, or an ultrasound imaging system. Alternatively or additionally, 2D images, e.g. MRI or X-ray projection images, may be acquired by tracking a landmark in the patient, whether it be a fiducial or other anatomical landmark. 3D-MRI or 3D-CT systems, however, may be less suitable to be used to deliver the updated data since such systems are usually associated with relatively long acquisition and reconstruction times, e.g. a high data latency. However, for slowly evolving cyclic or semi-cyclic motion, 3D-MRI and 3D-CT systems may alternatively, or additionally, be used to provide the updated data.

Preferably, the monitoring system 120 is also arranged to monitor the beam source(s) 102 to receive updated data on the current position(s) and/or alignment(s) of the at least one beam source 102. This data may be received by the monitoring system 120 from the beam supervising system 130 with low latency.

As previously stated, the biomechanical model 300 of the anatomical region 202 comprises mesh nodes 302 and mesh vertices 304, wherein each mesh node 302 is associated with a world-space coordinate x of the mesh node 302 and a state variable u that represents spatial-temporal displacement of the anatomical region 202, which state variable u may be a displacement field.

Consequently, in the graph network system 500 implemented in the control system 110, the trained biomechanical model 300 may be represented as a graph 400, in which the graph nodes 402 and the graph edges 404 are specified to represent the mesh nodes 302 and the mesh vertices 304, respectively, and the state variables, represented by the displacement field u, may be embedded in the graph edges. Additionally, as previously stated, internal forces within the biomechanical model 300 may be encoded as directional graph edge features between two adjacent graph nodes, representing the force exerted by one mesh node onto a neighbouring mesh node.

Consequently, the nodes, edges and global elements of the graph 400 may have the following features:

Node features (vᵢ)

| | |
|---|---|
| xᵢ | 3D coordinates of node vᵢ |
| x′ᵢ (optionally, for | First order derivative of xᵢ |
| second-order models) | |
| nᵢ | Node type |
| Nᵢ | Number of neighbours |

Edge features (eᵢⱼ)

| | |
|---|---|
| uᵢⱼ = xᵢ − xⱼ | Relative mesh-space displacement vector |
| | between node vᵢ and vⱼ. |
| \|uᵢⱼ\| | Norm of relative displacements |
| xᵢⱼ | Relative world-space displacement vector |
| | between node vᵢ and vⱼ. |
| fᵢⱼ | Internal forces |

Global features

| | |
|---|---|
| uₘₐₓ | Maximum allowed displacement (edge |
| | length) |
| V | Span of xᵢ |

The dynamic quantities of the system, i.e. the state variable u and its derivatives are embedded in the graph edges. In the processor 512, each graph network block 516 takes an input graph and transforms the dynamic quantities defined on the edges to produce an output graph.

The features received by each graph edge determine the physical interactions and properties from the biomechanical model that are encoded in the network.

The graph edges embed the relative displacement field u and its norm |u|. Additionally, internal forces fᵢⱼ in the model are encoded as directional edge features between two adjacent nodes, representing the force exerted by one node onto a neighbouring node.

Differential operators may be approximated by message-passing between adjacent graph nodes, which are embedded in the graph edges as sub-functions (ƒ^{M}). These functions take the edge features (e.g. derivatives and forces) and the sender and receiver node features (coordinates, material properties, etc.) as input, and output the higher-order derivatives of the nodes. These are in turn used to integrate the dynamic variable x.

The graph nodes may also embed functions (ƒ^{V}) which represent operations on the graph node features, independent of the messages passed between the graph nodes, e.g. transformations of the material properties.

$${e}_{ij}^{′ M} ← {f}^{M} ( {e}_{ij}^{M} , {v}_{i} , {v}_{j} )$$

$${v}_{i}^{′} ← {f}^{V} ( {v}_{i} , {\sum }_{j} {e}_{ij}^{′ M} , {\sum }_{j} {e}_{ij}^{′ W} )$$

Here, eᵢⱼ^{M} and eᵢⱼ^{W} are mesh and world edges, respectively.

The graph processing neural network 504 processes the graph node features, the graph edge features and the global features and may output the following:

Outputs

| | |
|---|---|
| x′ᵢ (optionally, for first-order models) | First-order derivative of xᵢ |
| x″ᵢ | Second-order derivative of xᵢ |
| Fᵢ | Field variable (e.g. stress) |

In the updater 506, the dynamic quantity xᵢ at time t+1 is then obtained by integrating the derivatives at time t. For first-order models, the dynamic quantity xᵢ is updated with the first derivative outputted by the graph processing neural network 504 and for the second-order models, x′ᵢ is embedded as a node feature and x″ᵢ is used to integrate the dynamic quantity xᵢ according the following:

$${x}_{i}^{t + 1} = {x}_{i}^{′} + {x}_{i}^{t} ( for first - order models )$$

$$or$$

$${x}_{i}^{t + 1} = {x}_{i}^{″} + 2 {x}_{i}^{t} - {x}_{i}^{t - 1} ( for second - order models )$$

thus providing, at output 508, an inferred future state M^{t+1} of the model having inferred features xᵢ^{t+1}, i.e. predicted future positions of the mesh nodes 302 of the model 300.

In operation of the radiotherapy system 100, a current state M^{t} of the trained model 300 is established. Preferably, establishing the current state M^{t} involves correlating the current state M^{t} with data on the current position and/or orientation in space of the anatomical region 202, i.e. real-world data. This data may preferably be provided by the monitoring system 120 and may for example be based on updated data on the position and/or orientation in space of a boundary area of the anatomical region 202, for example the envelope of the thorax of the radiotherapy patient, i.e. the outer contour (body surface) of the thorax, and/or the position and/or orientation in space of internal structures of the anatomical region, e.g. the position and/or orientation in space of one or a plurality of fiducial markers.

If the current state M^{t} does not correlate with the real-world data provided by the monitoring system 120, the current state M^{t} of the model 300 may be adjusted or updated to better represent the real-world data. If the correlation between the current state M^{t} and the real-world data is poor, e.g. below a predetermined limit, the radiotherapy treatment session may be aborted.

If the current state M^{t} is found to represent the real-world data, an inferred future state M^{t+1} of the trained model is provided by the graph network system 500 and the position and/or alignment of the at least one beam source 102 is controlled to track the irradiation target 204 based on the inferred future state M^{t+1} of the trained model 300.

The radiotherapy system 100 may be configured to provide an inferred future state M^{t+1} of the model 300 intermittently with a period within the range of 20 ms to 100 ms, e.g. every 50 ms.

According to the above-discussed embodiment, the current state M^{t} is correlated with the real-world data every time a current state M^{t} of the trained model 300 is established. In such a case, the monitoring system update rate should be no lower than the model update rate, e.g. every 50 ms. According to an alternative embodiment, the current state M^{t} may be correlated with the real-world data less often, e.g. every second or third time the current state M^{t} is established. In such an embodiment, the current state M^{t} may be based primarily on at least one of the previously inferred states of the model, M^{t-1}, M^{t-2}, M^{t-3} etc.

The radiotherapy system 100 according to the present disclosure allows a future position, alignment and/or deformation of the irradiation target 204 to be predicted, thus giving the beam supervising system 106 time to react on state changes of the irradiation target 204. Also, the radiotherapy system 100 allows the irradiation target 204 to be accurately tracked although no updated real-world data on the position, alignment and/or deformation of the irradiation target 204 is available.

Also, since the anatomical region 202 is modelled to include organs and tissue surrounding the irradiation target 204, movement and deformation of organs-at-risk can also be predicted, thus allowing the control system 110 to instruct the beam supervising system 106 to position and align the beam source(s) 102 such that irradiation of such organs can be avoided or at least minimised. Additionally or alternatively, if the inferred future state indicates that the radiation beam is at risk to intersect an organ-at-risk, the control system 110 may be configured to instruct the beam supervising system 106 to block the radiation beam or shut down the corresponding beam source. Blocking the radiation beam may be realised by applying a gating function to the beam source.

Additionally or alternatively, the control system 110 may be configured to instruct the beam supervising system 106 to block the radiation beam or to shut down the corresponding beam source if the inferred future state indicates that the irradiation target is about to venture outside the trajectory of the ionizing radiation beam, e.g. due to a situation in which the maximum range of the alignment system of the beams source may not be able to accommodate for the predicted movement of the irradiation target.

As previously stated, the trained model 300 may advantageously comprise variables characterising planned and/or maximum allowed radiation dose in or at each mesh node 302 or voxel, and also a variable characterising accumulated radiation dose in or at each mesh node 302. By receiving updated information on the position(s) and alignment(s) of the beam source(s), and thus information on where in the trained model 300 radiation energy is deposited, the control system 110 can, for each inferred future state M^{t+1} of the trained model, update the accumulated radiation dose in or at each mesh node 302, thus allowing the control system 110 to instruct the beam supervising system 106 to position and align the beam source(s) 102 such that the irradiation target 204 receives the planned radiation dose without the maximum allowed radiation dose for other parts of the anatomical region 202 is exceeded.

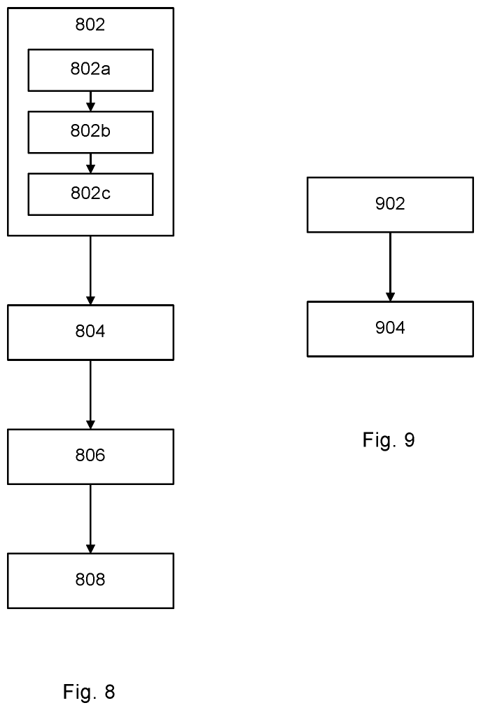

FIG. 8 illustrates an embodiment of a method of establishing a biomechanical model of an anatomical region of a radiotherapy patient for use in a radiotherapy system, e.g. for use in the above-discussed radiotherapy system 100.

The method comprises a step 802 of establishing a point-based or mesh-based model of the modelled anatomical region. The point-based or mesh-based model (300) may for example be the above-discussed mesh-based model 300.

The method further comprises a step 804 of associating static and/or dynamic features to points or mesh nodes of the point-based or mesh-based model. The points or mesh nodes of the point-based or mesh-based model may for example be the above-discussed mesh nodes 302 of the mesh-based model 300, The static and/or dynamic features comprise a world-space coordinate of the points or mesh nodes. For example, the world-space coordinate of the points or mesh nodes may be the above-discussed world-space coordinate x of the mesh nodes 302. The static and/or dynamic features also comprise a state variable representing spatial-temporal displacement of the anatomical region. For example, the state variable representing spatial-temporal displacement of the anatomical region may be the above-discussed state variable u representing spatial-temporal displacement of the anatomical region 202.

For each point or mesh node, the static and/or dynamic features also comprise at least one feature characterising body tissue in the anatomical region. The at least one feature characterising body tissue in the anatomical region may comprise at least one variable characterising interaction between the anatomical region and an ionizing radiation beam, e.g. at least one of: linear stopping power; relative stopping power; radiodensity and radiopacity.

The method further comprises a step 806 of representing the model as a graph in a graph neural network system. For example, the step 806 may comprise representing the above-discussed model 300 as a graph in the graph neural network system 500.

The method also comprises a step 808 of training the model on a cyclic or semi-cyclic motion of the anatomical region. For example, the step 808 may comprise training the model 300 on a cyclic or semi-cyclic motion of the anatomical region 202, as previously discussed.

The step 806 of representing the model as a graph in a graph neural network system may comprise establishing graph nodes, graph edges and graph global elements having features reflecting the static and dynamic variables of the biomechanical model, e.g. the features of the above-discussed model 300.

For example, when the above-discussed model 300 is represented as a graph in the above-discussed graph neural network system 500, the graph network system 500 may be configured to comprise an input 502, a graph processing neural network 504, an updater 506 and an output 508, as previously discussed, and the graph processing neural network 504 may comprise an encoder 510, a processor 512 and a decoder 514. The encoder 510 may be configured to receive a current state M^{t} of the mesh-based biomechanical model 300 from the input 502 and encode an input graph G⁰ from the current state M^{t} of the biomechanical model 300. The processor 512 may be configured to process the input graph G⁰ and provide an output graph G^{M} having updated node and edge embeddings. The decoder 514 may be configured to decode the output graph G^{M} extracting output features corresponding to the static and dynamical variables of the biomechanical model 300, and the updater 506 may be configured to update the output features to produce an inferred future state M^{t+1} of the mesh-based biomechanical model 300 and provide the inferred state M^{t+1} to the output 508.

The step 808 of training the model on a cyclic or semi-cyclic motion of the anatomical region may comprise applying a loss function on the inferred states M^{t+1} provided by the updater 506 and on a real-world dataset on an anatomical region corresponding to the modelled anatomical region. The real-world dataset may comprise at least one of: 4D computed tomography (4D-CT) scans, 4D magnetic resonance imaging (4D-MRI) and ultrasound imaging. The real-world dataset may comprise an evolution of images covering the modelled anatomical region during a respiratory cycle.

The point-based or mesh-based model may incorporate at least one of: a linear elasticity model and a hyperelasticity model adopted to the physical characteristics of the anatomical region.

The step 802 of establishing a point-based or mesh-based model of the modelled anatomical region may comprise a first sub-step 802a of dividing the anatomical region into at least a first anatomical sub-region displaying a first set of physical properties and a second anatomical sub-region displaying a second set of physical properties being different from the first set of physical properties; a second sub-step 802b of, for the first anatomical sub-region, assigning the point-based or mesh-based model a first point-based or mesh-based sub-model adopted to the first set of physical properties; and a third sub-step 802c of, for the second anatomical sub-region, assigning the point-based or mesh-based model a second point-based or mesh-based sub-model adopted to the second set of physical properties. In cases where the first and the second anatomical sub-region share a common boundary, the sub-steps 802b and 802c may comprise assigning the first and the second point-based or mesh-based sub-model common boundary conditions in the common boundary.

An embodiment of a computer implemented method of planning at least a part of a radiotherapy procedure to be carried out on an anatomical region of a radiotherapy patient is illustrated in FIG. 9. The method comprises a first step 902 of establishing a biomechanical model of the anatomical region according to the method discussed above with reference to FIG. 8, and a second step 904 of assigning, to each point or mesh node, at least one of planned dose and maximum allowed dose, thus allowing the model, during a subsequent radiotherapy session, to compare absorbed dose at different points or mesh nodes with planned or maximum allowed dose. As previously discussed, the radiotherapy system in which the model is implemented can then control dose delivery and beam alignment based on a difference between planned and absorbed dose such that the irradiation target receives a planned radiation dose without a maximum allowed radiation dose for other parts of the anatomical region is exceeded.

In the preceding description, various aspects of the apparatus according to the invention have been described with reference to the illustrative embodiment. For purposes of explanation, specific numbers, systems and configurations were set forth in order to provide a thorough understanding of the apparatus and its workings. However, this description is not intended to be construed in a limiting sense. Various modifications and variations of the illustrative embodiment, as well as other embodiments of the apparatus, which are apparent to persons skilled in the art to which the disclosed subject matter pertains, are deemed to lie within the scope of the present invention a defined by the following claims.

1. A method of establishing a biomechanical model of an anatomical region of a radiotherapy patient for use in a radiotherapy system, the method comprising the steps of:
establishing a point-based or mesh-based model of the anatomical region;
associating static and/or dynamic features to points or mesh nodes of the point-based or mesh-based model, the features comprising a world-space coordinate (x) of the points or mesh nodes and state variable (u) representing spatial-temporal displacement of the anatomical region and, for each point or mesh node, at least one feature characterising body tissue in the anatomical region;
representing the model as a graph in a graph neural network system; and
training the model on a cyclic or semi-cyclic motion of the anatomical region.

2. The method according to claim 1, wherein the at least one feature characterising body tissue in the anatomical region comprises at least one variable characterising interaction between the anatomical region and an ionizing radiation beam.

3. The method according to claim 2, wherein the at least one feature characterising interaction between the anatomical region and the ionizing radiation beam comprises at least one of: linear stopping power; relative stopping power;
radiodensity and radiopacity.

4. The method according to claim 1, wherein the step of representing the model as a graph in a graph neural network system comprises establishing graph nodes, graph edges and graph global elements having features reflecting the static and dynamic variables of the biomechanical model;
wherein the graph network system comprises an input, a graph processing neural network, an updater and an output, and wherein the graph processing neural network comprises an encoder, a processor and a decoder;
wherein the encoder is configured to receive a current state (M^{t}) of the mesh-based biomechanical model from the input and encode an input graph (G⁰) from the current state (M^{t}) of the biomechanical model;
wherein the processor is configured to process the input graph (G⁰) and provide an output graph (G^{M}) having updated node and edge embeddings;
wherein the decoder is configured to decode the output graph (G^{M}) extracting output features corresponding to the static and dynamical variables of the biomechanical model; and
wherein the updater is configured to update the output features to produce an inferred future state (M^{t+1}) of the mesh-based biomechanical model and provide the inferred state (M^{t+1}) to the output.

5. The method according to claim 4, wherein the step of training the model on a cyclic or semi-cyclic motion of the anatomical region comprises applying a loss function on the inferred states (M^{t+1}) provided by the updater and on a real-world dataset on an anatomical region corresponding to the modelled anatomical region.

6. The method according to claim 5, wherein the real-world dataset comprises at least one of: 4D computed tomography (4D-CT) scans, 4D magnetic resonance imaging (4D-MRI) and ultrasound imaging.

7. The method according to claim 5, wherein the real-world dataset comprises an evolution of images covering the modelled anatomical region during a respiratory cycle.

8. The method according to claim 1, wherein the point-based or mesh-based model incorporates at least one of a linear elasticity model and a hyperelasticity model adopted to the physical characteristics of the anatomical region.

9. The method according to claim 1,
comprising the steps of:
dividing the anatomical region into at least a first anatomical sub-region displaying a first set of physical properties and a second anatomical sub-region displaying a second set of physical properties being different from the first set of physical properties;
for the first anatomical sub-region, assigning the point-based or mesh-based model a first point-based or mesh-based sub-model adopted to the first set of physical properties; and
for the second anatomical sub-region, assigning the point-based or mesh-based model a second point-based or mesh-based sub-model adopted to the second set of physical properties.

10. The method according claim 9, wherein the first and the second anatomical sub-region share a common boundary, the method comprising the step of assigning the first and the second point-based or mesh-based sub-model common boundary conditions in the common boundary.

11. A computer implemented method of planning at least a part of a radiotherapy procedure to be carried out on an anatomical region of a radiotherapy patient, comprising:
establishing a biomechanical model of the anatomical region according to any one of claims 1-10; and
assigning, to each point or mesh node, at least one of: planned dose and maximum allowed dose.

12. A radiotherapy system comprising:
at least one beam source arranged to provide an ionizing radiation beam to an irradiation target located in an anatomical region;
a beam supervising system configured to control position and/or alignment of the at least one beam source; and
a monitoring system arranged to monitor the anatomical region,
wherein:
a control system comprising a computer-implemented graph network system in which a model of the anatomical region, trained on cyclic or semi-cyclic motion of the anatomical region, is implemented;
the graph network system being configured to establish a current state (M^{t}) of the trained model based on data on a current position and/or on a current orientation in space of the anatomical region received from the monitoring system, and
being configured to establish an inferred future state (M^{t+1}) of the trained model based on the current state (M^{t}); and
the control system being configured to instruct the beam supervising system to control the ionizing radiation beam of the at least one beam source based on the inferred future state (M^{t}).

13. The radiotherapy system according to claim 12, wherein the control system is configured to instruct the beam supervising system to control the ionizing radiation beam of the at least one beam source based on the inferred future state (M^{t+1}) to bring the least one beam source to track the irradiation target.

14. The radiotherapy system according to claim 12, wherein the control system is configured to instruct the beam supervising system to control the ionizing radiation beam of the at least one beam source based on the inferred future state (M^{t+1}) to block the ionizing radiation beam of the at least one beam source or to shut down the at least one beam source should the inferred future state (M^{t+1}) indicate that the irradiation target is about to venture outside the trajectory of the ionizing radiation beam.

15. The radiotherapy system according to claim 12, wherein the control system is configured to instruct the beam supervising system to control the ionizing radiation beam of the at least one beam source based on the inferred future state (M^{t+1}) to block the ionizing radiation beam of the at least one beam source or to shut down the at least one beam source should the inferred future state (M^{t+1}) indicate that the trajectory of the ionizing radiation beam is at risk of intersecting an organ-at-risk.

16. The radiotherapy system according to claim 12, wherein the trained model is a point-based or mesh-based model comprising static and/or dynamic features associated with points or mesh nodes of the point-based or mesh-based model, the features comprising a world-space coordinate (x) of the points or mesh nodes and state variable (u) representing spatial-temporal displacement of the anatomical region.

17. The radiotherapy system according to claim 12, wherein the graph network system is configured to represent the trained model as a graph comprising graph nodes, graph edges and graph global elements having features reflecting the static and dynamic variables of the trained model.

18. The radiotherapy system according to claim 12, wherein the graph network system is configured to correlate the established current state (M^{t}) of the trained model with said data received from the monitoring system.

19. The radiotherapy system according to claim 18, wherein the monitoring system is configured to monitor at least one of: a position and/or an orientation in space of a boundary area of the anatomical region; and a position and/or an orientation in space of a fiducial marker in the anatomical region.

20. The radiotherapy system according to claim 12,
wherein said variables comprise at least one variable characterising interaction between the anatomical region and an ionizing radiation beam, and at least one variable characterising at least one of: planned radiation dose; maximum allowed radiation dose; and accumulated radiation dose,
wherein the graph network system, for each inferred future state (M^{t+1}) of the trained model, is configured to update the variable characterising the accumulated radiation dose, and
wherein the control system is configured to instruct the beam supervising system to control position and/or alignment of the at least one beam source such that the irradiation target receives a planned radiation dose without a maximum allowed radiation dose for other parts of the anatomical region is exceeded.

21. The radiotherapy system according to claim 12, wherein the monitoring system is arranged to monitor the beam supervising system.

22. The radiotherapy system according to claim 12, wherein the trained model of the anatomical region has been established according to any one of claims 1-10.
